(19) United States
(12) Patent Application Publication
(10) Pub. No.: US 2013/0345779 A1
(43) Pub. Date: Dec. 26, 2013

(54) TWO DIMENSIONAL AND ONE DIMENSIONAL FIELD ELECTROPORATION

(71) Applicant: THE REGENTS OF THE UNIVERSITY OF CALIFORNIA, Oakland, CA (US)

(72) Inventors: Elad MAOR, Berkeley, CA (US); Antoni IVORRA, Berkeley, CA (US); Boris RUBINSKY, El Cerrito, CA (US); Francois FERNARD, Besancon (FR); Liel RUBINSKY, El Cerrito, CA (US); Alex GOLBERG, Jerusalem (IL)

(21) Appl. No.: 13/722,660

(22) Filed: Dec. 20, 2012

Related U.S. Application Data
(60) Provisional application No. 61/585,972, filed on Jan. 12, 2012.

(51) Int. Cl. A61N 1/32

(52) U.S. Cl. CPC A61N 1/327
USPC 607/115

(57) ABSTRACT

The shape and relative positions of two or more electrodes connected to a shaped metal surface are adjusted. By adjusting the shape and position of the electrodes, as well as the shape of the metal surface, the shape of the electrical field generated from the metal surface is precisely defined. The metal surface is brought into contact with cells and the defined electrical field provides reversible or irreversible electroporation to cells in a precisely defined area. The metal surface may be comprised of copper, silver, gold or other conductive material and combinations thereof and the voltage, wattage and duration of electricity applied to the electrodes can be varied to obtain a desired result.

### CROSS-REFERENCE

This application claims the benefit of U.S. Provisional Application No. 61/585,972, filed Jan. 12, 2012, which application is incorporated herein by reference.

### FIELD OF THE INVENTION

The invention relates generally to devices, systems and methods for carrying out electroporation of cells and particularly irreversible electroporation which is carried out using a metal surface to control the shape of the electrical field generated and directed at target cells.

### BACKGROUND OF THE INVENTION

In many medical procedures, such as the treatment of benign or malignant tumors, it is important to be able to ablate the undesirable tissue in a controlled and focused way without affecting the surrounding desirable tissue. Over the years, a large number of minimally invasive methods have been developed to selectively destroy specific areas of undesirable tissues as an alternative to resection surgery. There are a variety of techniques with specific advantages and disadvantages, which are indicated and contraindicated for various applications. For example, cryosurgery is a low temperature minimally invasive technique in which tissue is frozen on contact with a cryogen cooled probe inserted in the undesirable tissue (Rubinsky, B., ed. Cryosurgery. Annu. Rev. Biomed. Eng. Vol. 2. 2000. 157-187.). The area affected by low temperature therapies, such as cryosurgery, can be easily controlled through imaging. However, the probes are large and difficult to use. Non-selective chemical ablation is a technique in which chemical agents such as ethanol are injected in the undesirable tissue to cause ablation (Shiina, S., et al., Percutaneous ethanol injection therapy for hepatocellular carcinoma: results in 146 patients. AJR, 1993. 160: p. 1023-8). Non-selective chemical therapy is easy to apply. However, the affected area cannot be controlled because of the local blood flow and transport of the chemical species. Elevated temperatures are also used to ablate tissue. Focused ultrasound is a high temperature non-invasive technique in which the tissue is heated to coagulation using high-intensity ultrasound beams focused on the undesirable tissue (Lynn, J. G., et al., A new method for the generation of use of focused ultrasound in experimental biology. J. Gen Physiol., 1942. 26: p. 179-93; Foster, R. S., et al., High-intensity focused ultrasound in the treatment of prostatic disease. Eur. Urol., 1993. 23: p. 44-7). Electrical currents are also commonly used to heat tissue. Radiofrequency ablation (RF) is a high temperature minimally invasive technique in which an active electrode is introduced in the undesirable tissue and a high frequency alternating current of up to 500 kHz is used to heat the tissue to coagulation (Organ, L. W., Electrophysiological principles of radiofrequency lesion making. Appl. Neurophysiol., 1976. 39: p. 69-76). In addition to RF heating traditional Joule heating methods with electrodes inserted in tissue and dc or ac currents are also common, (Erez, A., Shitzer, A. (Controlled destruction and temperature distribution in biological tissue subjected to monoactive electrocoagulation) J. Biomech. Eng. 1980:102(1):42-9). Interstitial laser coagulation is a high temperature thermal technique in which tumors are slowly heated to temperatures exceeding the threshold of protein denaturation using low power lasers delivered to the tumors by optical fibers (Bown, S. G., Phototherapy of tumors. World. J. Surgery, 1983. 7: p. 700-9). High temperature thermal therapies have the advantage of ease of application. The disadvantage is the extent of the treated area is difficult to control because blood circulation has a strong local effect on the temperature field that develops in the tissue. The armamentarium of surgery is enhanced by the availability of the large number of minimally invasive surgical techniques in existence, each with their own advantages and disadvantages and particular applications. This document discloses another minimally invasive surgical technique for tissue ablation, irreversible electroporation. We will describe the technique, evaluate its feasibility through mathematical modeling and demonstrate the feasibility with in vivo experimental studies.

Electroporation is defined as the phenomenon that makes cell membranes permeable by exposing them to certain electric pulses (Weaver, J. C. and Y. A. Chizmadzhev, Theory of electroporation: a review. Bioelectrochem. Bioenerg., 1996. 41: p. 135-60). Electroporation pulses are defined as those electrical pulses that through a specific combination of amplitude, shape, time length and number of repeats produce no other substantial effect on biological cells than the permeabilization of the cell membrane. The range of electrical parameters that produce electroporation is bounded by: a) parameters that have no substantial effect on the cell and the cell membrane, b) parameters that cause substantial thermal effects (Joule heating) and c) parameters that affect the interior of the cell, e.g. the nucleus, without affecting the cell membrane. Joule heating, the thermal effect that electrical currents produce when applied to biological materials is known for centuries. It was noted in the previous paragraph that electrical thermal effects which elevate temperatures to values that damage cells are commonly used to ablate undesirable tissues. The pulse parameters that produce thermal effects are longer and/or have higher amplitudes than the electroporation pulses whose only substantial effect is to permeabilize the cell membrane.

There are a variety of methods to electrically produce thermal effects that ablate tissue. These include RF, electrode heating, and induction heating. Electrical pulses that produce thermal effects are distinctly different from the pulses which produce electroporation. The distinction can be recognizing through their effect on cells and their utility. The effect of the thermal electrical pulses is primarily on the temperature of the biological material and their utility is in raising the temperature to induce tissue ablation through thermal effects.

The effect of the electroporation parameters is primarily on the cell membrane and their utility is in permeabilizing the cell membrane for various applications. Electrical parameters that only affect the interior of the cell, without affecting the cell membrane were also identified recently. They are normally referred to as “nanosecond pulses”. It has been shown that high amplitude, and short (substantially shorter than electroporation pulses—nanoseconds versus millisecond) length pulses can affect the interior of the cell and in particular the nucleus without affecting the membrane. Studies on nanosecond pulses show that they are “distinctly different than electroporation pulses” (Beebe S J. Fox P M. Rec L J. Somers K. Stark R H. Schoenbach K H. Nanosecond pulsed electric field (nsPEF) effects on cells and tissues: apoptosis induction and tumor growth inhibition. PPPS-2001 Pulsed Power Plasma Science 2001. 28th IEEE International Conference on Plasma Science and 13th IEEE International Pulsed Power Conference. Digest of Technical Papers (Cat. No. 01CH37251). IEEE. Part vol. 1, 2001, pp. 211-15 vol. 1. Piscataway, N.J., USA. Several applications have been identified for nano-second pulses. One of them is for tissue ablation through an effect on the nucleus (Schoenbach, K. H., Beebe, S. J., Buescher, K. S. Method and apparatus for intracellular electro-manipulation U.S. Patent Application Pub No. US 2002/0010491 A1, Jan. 24, 2002). Another is to regulate genes in the cell interior, (Gunderson, M. A. et al. Method for intracellular modification within living cells using pulsed electrical fields—regulate gene transcription and entering intracellular US Patent application 2003/0170898 A1, Sep. 11, 2003). Electrical pulses that produce intracellular effects are distinctly different from the pulses which produce electroporation. The distinction can be recognizing through their effect on cells and their utility. The effect of the intracellular electrical pulses is primarily on the intracellular contents of the cell and their utility is in manipulating the intracellular contents for various uses—including ablation. The effect of the electroporation parameters is primarily on the cell membrane and their utility is in permeabilizing the cell membrane for various applications, which will be discussed in greater detail later.

Electroporation is known for over half a century. It was found that as a function of the electrical parameters, electroporation pulses can have two different effects on the permeability of the cell membrane. The permeabilization of the membrane can be reversible or irreversible as a function of the electrical parameters used. In reversible electroporation the cell membrane reseals a certain time after the pulses cease and the cell survives. In irreversible electroporation the cell membrane does not reseal and the cell lyses. A schematic diagram showing the effect of electrical parameters on the cell membrane permeabilization (electroporation) and the separation between: no effect, reversible electroporation and irreversible electroporation is shown in FIG. 1 (Dev, S. B., Rabussay, D. P., Widera, G., Hofmann, G. A., Medical applications of electroporation, IEEE Transactions of Plasma Science, Vol 28 No 1, February 2000, pp 206-223) Dielectric breakdown of the cell membrane due to an induced electric field, irreversible electroporation, was first observed in the early 1970s (Neumann, E. and K. Rosenheck, Permeability changes induced by electric impulses in vesicular membranes. J. Membrane Biol., 1972. 10: p. 279-290; Crowley, J. M., Electrical breakdown of biomolecular lipid membranes as an electromechanical instability. Biophysical Journal, 1973. 13: p. 711-724; Zimmermann, U., J. Vienken, and G. Pilwat, Dielectric breakdown of cell membranes. Biophysical Journal, 1974. 14(11): p. 881-899). The ability of the membrane to reseal, reversible electroporation, was discovered separately during the late 1970s (Kinosita Jr, K. and T. Y. Tsong, Hemolysis of human erythrocytes by a transient electric field. Proc. Natl. Acad. Sci. USA, 1977. 74(5): p. 1923-1927; Baker, P. F. and D. E. Knight, Calcium-dependent exocytosis in bovine adrenal medullary cells with leaky plasma membranes. Nature, 1978. 276: p. 620-622; Gauger, B. and F. W. Bentrup, A Study of Dielectric Membrane Breakdown in the Fucus Egg, J. Membrane Biol., 1979. 48(3): p. 249-264).

The mechanism of electroporation is not yet fully understood. It is thought that the electrical field changes the electrochemical potential around a cell membrane and induces instabilities in the polarized cell membrane lipid bilayer. The unstable membrane then alters its shape forming aqueous pathways that possibly are nano-scale pores through the membrane, hence the term “electroporation” (Chang, D. C., et al., Guide to Electroporation and Electrofusion. 1992, San Diego, Calif.: Academic Press, Inc.). Mass transfer can now occur through these channels under electrochemical control. Whatever the mechanism through which the cell membrane becomes permeabilized, electroporation has become an important method for enhanced mass transfer across the cell membrane.

The first important application of the cell membrane permeabilizing properties of electroporation is due to Neumann (Neumann, E., et al., Gene transfer into mouse lyoma cells by electroporation in high electric fields. J. EMBO, 1982. 1: p. 841-5). He has shown that by applying reversible electroporation to cells it is possible to sufficiently permeabilize the cell membrane so that genes, which are macromolecules that normally are too large to enter cells, can after electroporation enter the cell. Using reversible electroporation electrical parameters is crucial to the success of the procedure, since the goal of the procedure is to have a viable cell that incorporates the gene.

Following this discovery electroporation became commonly used to reversible permeabilize the cell membrane for various applications in medicine and biotechnology to introduce into cells or to extract from cells chemical species that normally do not pass, or have difficulty passing across the cell membrane, from small molecules such as fluorescent dyes, drugs and radioactive tracers to high molecular weight molecules such as antibodies, enzymes, nucleic acids, HMW dextrans and DNA. It is important to emphasize that in all these applications electroporation needs to be reversible since the outcome of the mass transport requires for the cells to be alive after the electroporation.

Following work on cells outside the body, reversible electroporation began to be used for permeabilization of cells in tissue. Heller, R., R. Gilbert, and M. J. Jaroszeski, Clinical applications of electrochemotherapy. Advanced drug delivery reviews, 1999. 35: p. 119-129. Tissue electroporation is now becoming an increasingly popular minimally invasive surgical technique for introducing small drugs and macromolecules into cells in specific areas of the body. This technique is accomplished by injecting drugs or macromolecules into the affected area and placing electrodes into or around the targeted tissue to generate reversible permeabilizing electric field in the tissue, thereby introducing the drugs or macromolecules into the cells of the affected area (Mir, L. M., Therapeutic perspectives of in vivo cell electropermeabilization. Bioelectrochemistry, 2001. 53: p. 1-10).

The use of electroporation to ablate undesirable tissue was introduced by Okino and Mohri in 1987 and Mir et al. in 1991. They have recognized that there are drugs for treatment of cancer, such as bleomycin and cys-platinum, which are very effective in ablation of cancer cells but have difficulties penetrating the cell membrane. Furthermore, some of these drugs, such as bleomycin, have the ability to selectively affect cancerous cells which reproduce without affecting normal cells that do not reproduce. Okino and Mori and Mir et al. separately discovered that combining the electric pulses with an impermeant anticancer drug greatly enhanced the effectiveness of the treatment with that drug (Okino, M. and H. Mohri, Effects of a high-voltage electrical impulse and an anticancer drug on in vivo growing tumors. Japanese Journal of Cancer Research, 1987. 78(12): p. 1319-21; Mir, L. M., et al., Electrochemotherapy potentiation of antitumour effect of bleomycin by local electric pulses. European Journal of Cancer, 1991. 27: p. 68-72). Mir et al. soon followed with clinical trials that have shown promising results and coined the treatment electrochemotherapy (Mir, L. M., et al., Electrochemotherapy, a novel antitumor treatment: first clinical trial. C. R. Acad. Sci., 1991. Ser. III 313(613-8)).

Currently, the primary therapeutic in vivo applications of electroporation are antitumor electrochemotherapy (ECT), which combines a cytotoxic nonpermeant drug with permeabilizing electric pulses and electrogenetherapy (EGT) as a form of non-viral gene therapy, and transdermal drug delivery (Mir, L. M., Therapeutic perspectives of in vivo cell electropermeabilization. Bioelectrochemistry, 2001. 53: p. 1-10). The studies on electrochemotherapy and electrogenetherapy have been recently summarized in several publications (Jaroszeski, M. J., et al., In vivo gene delivery by electroporation. Advanced applications of electrochemistry, 1999. 35: p. 131-137; Heller, R., R. Gilbert, and M. J. Jaroszeski, Clinical applications of electrochemotherapy. Advanced drug delivery reviews, 1999. 35: p. 119-129; Mir, L. M., Therapeutic perspectives of in vivo cell electropermeabilization. Bioelectrochemistry, 2001. 53: p. 1-10; Davalos, R. V., Real Time Imaging for Molecular Medicine through electrical Impedance Tomography of Electroporation, in Mechanical Engineering. 2002, University of California at Berkeley: Berkeley. p. 237). A recent article summarized the results from clinical trials performed in five cancer research centers. Basal cell carcinoma (32), malignant melanoma (142), adenocarcinoma (30) and head and neck squamous cell carcinoma (87) were treated for a total of 291 tumors (Mir, L. M., et al., Effective treatment of cutaneous and subcutaneous malignant tumours by electrochemotherapy. British Journal of Cancer, 1998. 77(12): p. 2336-2342).

Electrochemotherapy is a promising minimally invasive surgical technique to locally ablate tissue and treat tumors regardless of their histological type with minimal adverse side effects and a high response rate (Dev, S. B., et al., Medical Applications of Electroporation. IEEE Transactions on Plasma Science, 2000. 28(1): p. 206-223; Heller, R., R. Gilbert, and M. J. Jaroszeski, Clinical applications of electrochemotherapy. Advanced drug delivery reviews, 1999. 35: p. 119-129). Electrochemotherapy, which is performed through the insertion of electrodes into the undesirable tissue, the injection of cytotoxic dugs in the tissue and the application of reversible electroporation parameters, benefits from the ease of application of both high temperature treatment therapies and non-selective chemical therapies and results in outcomes comparable of both high temperature therapies and non-selective chemical therapies.

In addition, because the cell membrane permeabilization electrical field is not affected by the local blood flow, the control over the extent of the affected tissue by this mode of ablation does not depend on the blood flow as in thermal and non-selective chemical therapies. In designing electroporation protocols for ablation of tissue with drugs that are incorporated in the cell and function in the living cells it was important to employ reversible electroporation; because the drugs can only function in a living cell. Therefore, in designing protocols for electrochemotherapy the emphasize was on avoiding irreversible electroporation. The focus of the entire field of electroporation for ablation of tissue was on using reversible pulses, while avoiding irreversible electroporation pulses, that can cause the incorporation of selective drugs in undesirable tissue to selectively destroy malignant cells. Electrochemotherapy which employs reversible electroporation in combination with drugs, is beneficial due to its selectivity however, a disadvantage is that by its nature, it requires the combination of chemical agents with an electrical field and it depends on the successful incorporation of the chemical agent inside the cell.

An important concern in the studies of electrochemotherapy and electrogenetherapy in living tissue is the effect of electroporation on blood flow. Martin et al., have found that when reversible electroporation is used for introducing genes into cells on the blood vessel wall the blood vessels remain intact and their response to stimuli where indistinguishable from those of control vessels (Martin, J. B., Young, J. L., Benoit, J. N., Dean, D. A., Gene transfer to intact Mesenteric arteries by electroporation, Journal of vascular research, 2000, Vol 37:372-380). Ivanusa et al have found using MRI that with certain electroporation pulses, which appear to be in the irreversible electroporation range, that the electroporation transiently but significantly reduced tumor blood flow (Ivanusa, T, Berays, K., Cemazar, M., Jevtic, V, Demsar, F., Sersa, G. MRI macromolecular contrast agents as indicators of changed tumor blood flow, Radiol. Oncol. 2001; 35(2): 139-47). These findings are very different from those described here.

Sersa et al performed studies whose goal was to determine the effect of electrochemotherapy, reversible elctroporation with bleomycin or cisplatin, on tumor blood flow (Sersa, G., Sentjurc, M., Ivanusa, T, Berays, K., Kotnik, V, Coer, A., Swartz, H. M., Cemazar, M. Reduced blood flow and axygenation in SA-1 tumours after electrochemotherapy with cisplatin, Br. J. Cancer, 2002: 87(9):1047-54) (Sersa, G., Cemazar, M., Miklavcic, D. Tumor blood flow modifying effects of electrochemotherapy: a potential targeted mechanism radiol. Oncol 2003: 37(1): 43-8). In the first of the papers they report reduced blood flow that persisted for several days when using reversible electroporation with cisplatin. In the second paper they report complete shut down of blood flow after 24 hours when using reversible electroporation with bleomycin and 50% reduction in blood flow when using reversible electroporation with cisplatin.

### SUMMARY OF THE INVENTION

A method of directing an electric field at a target area of cells is disclosed. The method is carried out by first determining the shape of the area of cells to be targeted. A shaped metal surface is then positioned in contact with the targeted area of cells. Two or more electrodes are then positioned on the shaped metal surface. The positioning and shape of the electrodes is taken into consideration. In terms of the shape of electric field which will be generated or emitted from the metal surface when a charge is generated. An electrical charge differential is generated between the electrodes and based on the shape and positioning of the electrodes as well as the shape and positioning of the metal surface a precisely shaped electrical field is generated and brought into contact with the targeted area of cells. These cells can be subjected to reversible or irreversible electroporation.

An aspect of the invention is shaping a metal surface and shaping and positioning electrodes thereon in order to precisely shape an electric field generated from the metal surface.

These and other objects, advantages, and features of the invention will become apparent to those persons skilled in the art upon reading the details of the device, system and methodology as more fully described below.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures:

FIG. 1 includes photo 1A of a typical cuvette for cell electroporation and FIG. 1B shows a suspension and an electrical field generated around cells.

FIG. 2 shows a schematic drawing of cells trapped in a hole in a dielectric membrane with an electrical field developed between upper and lower electrodes.

FIG. 3 shows a device comprised of an electrically conductive metal surface having a layer of cells positioned thereon.

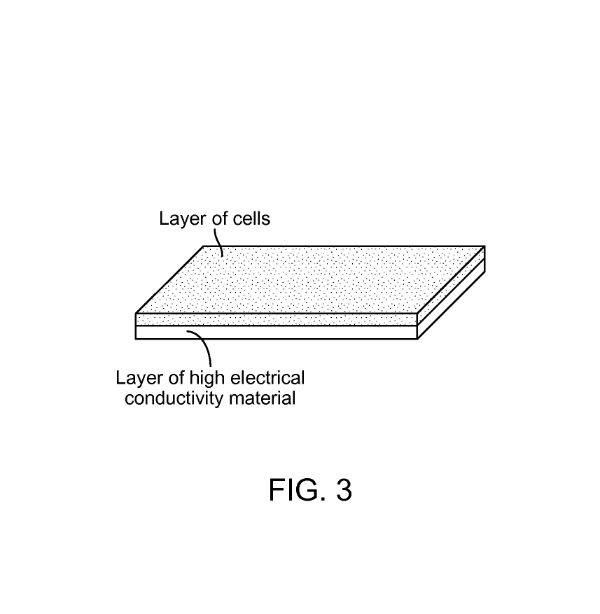

FIG. 4 shows a device comprised of a layer of electrically conductive material having a surface with a layer of cells positioned thereon and an upper layer of electrically conductive material which has greater electric conductivity as compared to the lower layer.

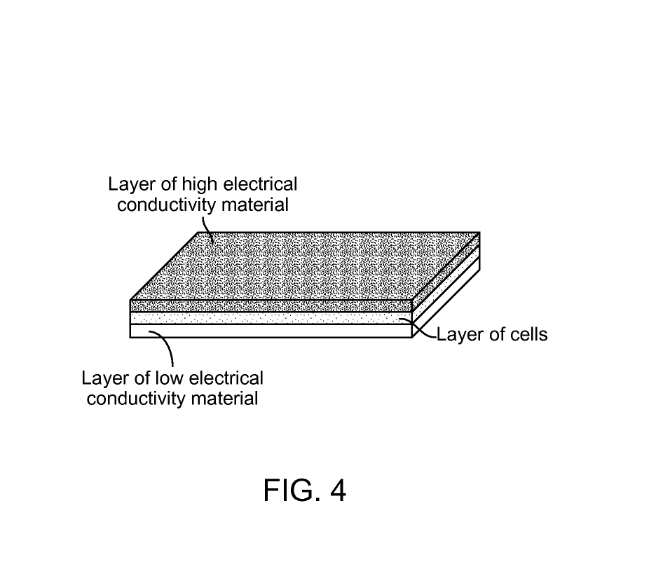

FIG. 5 shows a computer generated graph of an electric field generated around two electrodes immersed in a saline solution.

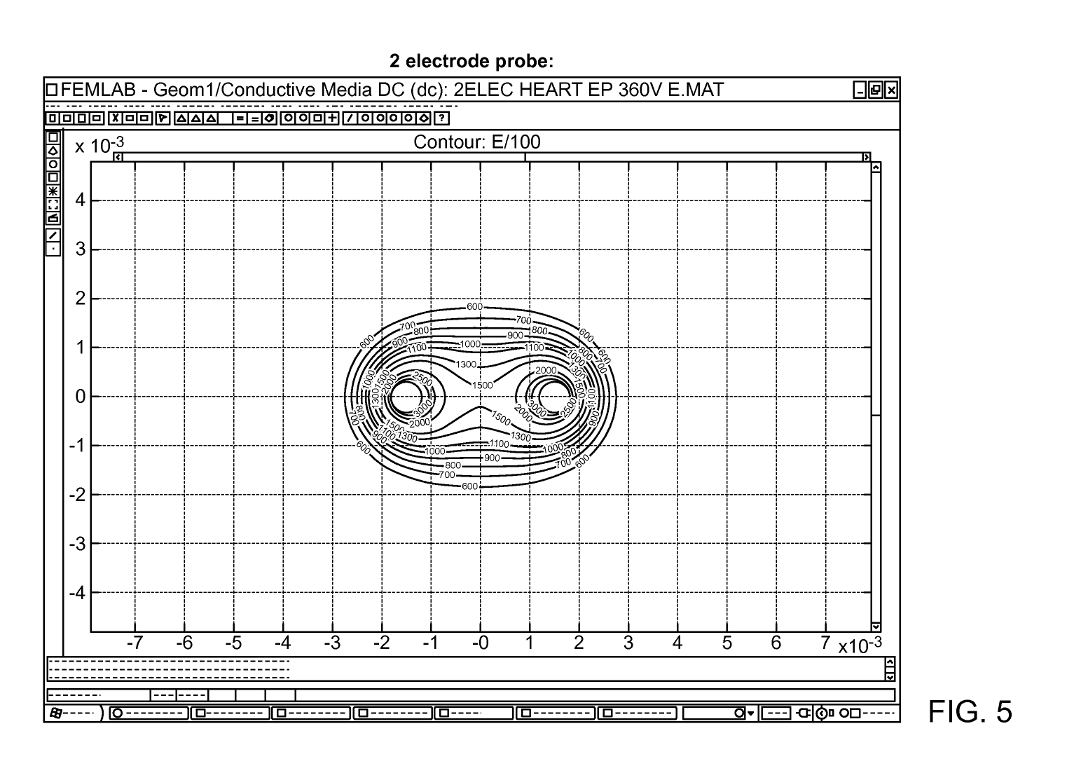

FIG. 6 shows a photograph of a layer of cells subjected to irreversible electroporation with the cells in the dark shape representing the cells subjected to electroporation by the shaped field.

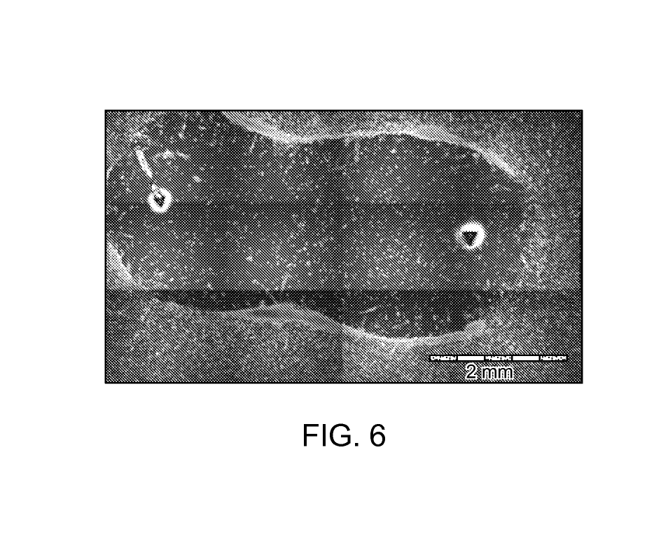

FIG. 7 is a graph showing different levels of electroporation.

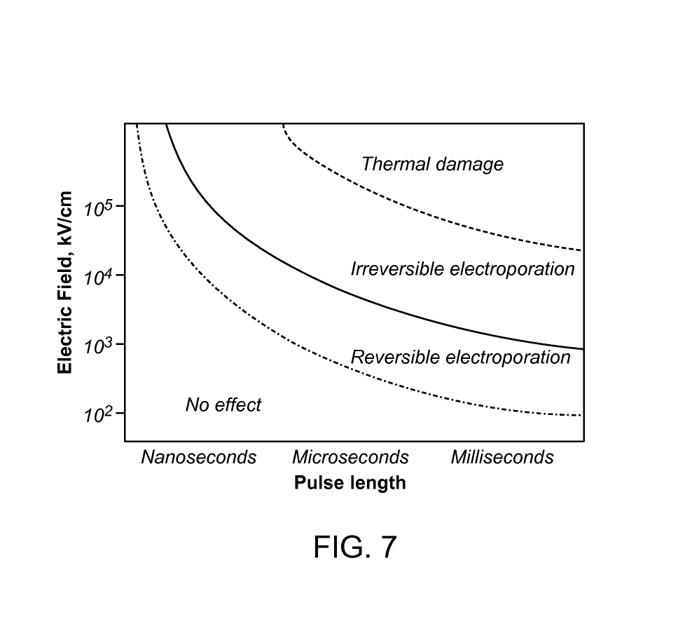

FIG. 8 includes photo (a), diagram (b) and graphs (c) and (d).

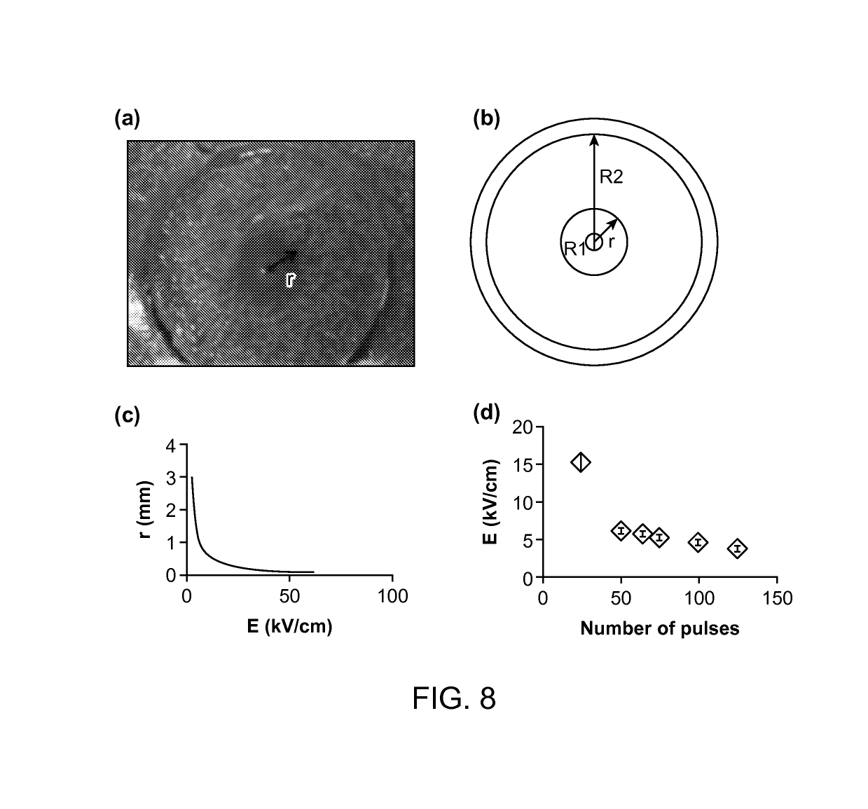

### DETAILED DESCRIPTION OF THE INVENTION

Before the present devices, systems, and methods of treatment and use are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “an electrode” includes a plurality of such electrodes and reference to “the pulse” includes reference to one or more pulses and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The term “reversible electroporation” encompasses permeabilization of the cell membrane through the application of electrical pulses across the cell. In “reversible electroporation” the permeabilization of the cell membrane ceases after the application of the pulse and the cell membrane permeability reverts to normal. The cell survives “reversible electroporation.” It is used as a means for introducing chemicals, DNA, or other materials into cells.

The term “irreversible electroporation” also encompasses the permeabilization of the cell membrane through the application of electrical pulses across the cell. However, in “irreversible electroporation” the permeabilization of the cell membrane does not cease after the application of the pulse and the cell membrane permeability does not revert to normal. The cell does not survive “irreversible electroporation” and the cell death is caused by the disruption of the cell membrane and not merely by internal perturbation of cellular components. Openings in the cell membrane are created and/or expanded in size resulting in a fatal disruption in the normal controlled flow of material across the cell membrane. The cell membrane is highly specialized in its ability to regulate what leaves and enters the cell. Irreversible electroporation destroys that ability to regulate in a manner such that the cell can not compensate and as such the cell dies.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

The mathematical solution to the electric field equation in cylindrical coordinates, has suggested to us a new experimental methodology and device for reducing experimental effort in designing electroporation protocols. Using a new cylindrical electroporation system, we show, with an Escherichia coli cell model, how key electroporation parameters emerge precisely from single experiments rather than through interpolation from numerous experiments in the conventional Cartesian electroporation system.

The permeabilization of the cell membrane using electric fields applied across the membrane is known as electroporation (Neumann et al. 1982) or electropermeabilization (Stopper et al. 1985). Electroporation is reversible when cells survive the electropermeabilization and irreversible when they do not. Reversible electroporation is commonly used in biotechnology and medicine for such applications as gene or drug delivery into cells (Dev et al. 2000). Irreversible electroporation is important for non-thermal sterilization in the food industry, biotechnology and medicine, and for tissue ablation in medicine (Pakhomov et al. 2010; Rubinsky 2010).

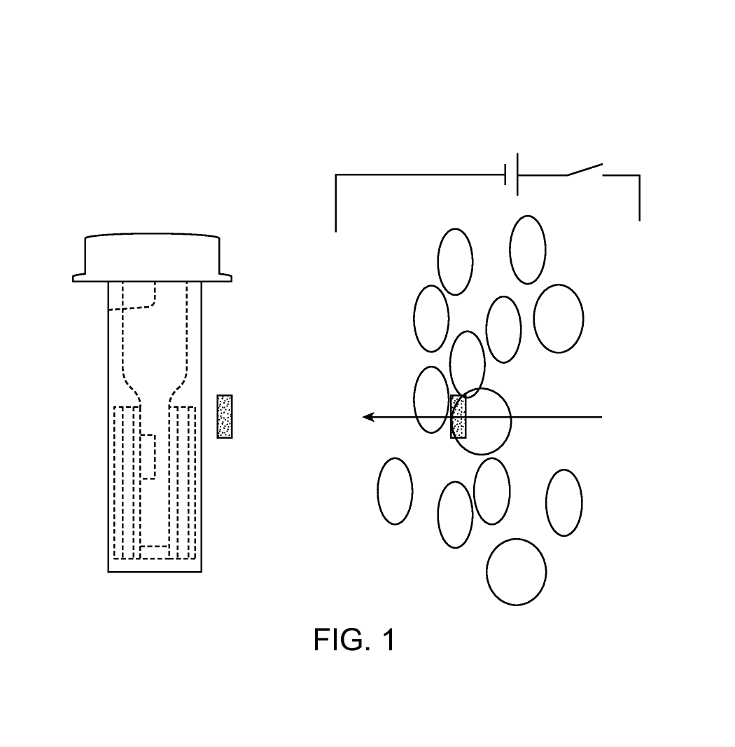

The outcome of an electroporation protocol, whether reversible or irreversible, depends on the parameters of the electric field such as strength, pulse length, number of pulses, time interval between pulses, frequency; on solution composition, pH, temperature and on cell type, shape and size. Because electroporation depends on so many parameters, designing optimal electroporation protocols requires tedious and lengthy efforts. To illustrate the complexity of protocol design, FIG. 1 shows a theoretical curve adapted from (Dev et al. 2000), which correlates electric field strength, single pulse length and the biophysical phenomenon that occurs when the particular parameters are applied across a cell. One of the most important features of the figure is the line that separates between the reversible and irreversible electroporation domains, which is critical in designing optimal electroporation protocols. In optimal reversible electroporation it is desirable to be close to and below that line while in optimal irreversible electroporation it is desirable to be close to and above that line. Conventional methods for the systematic development of optimal electroporation protocols employ experimental systems made of two parallel electrodes, bounding the media of interest, in a one-dimensional Cartesian configuration e.g. (Sale and Hamilton 1967; Hamilton and Sale 1967). The solution to the simple Laplace equation (∇²φ=0; where φ is the potential) for a homogeneous Cartesian system, subject to constant voltage boundary conditions on the electrodes, V₂ and V₁, gives an expression for the electric field between the planar electrodes. It is,

$$\frac{( {V}_{2} - {V}_{1} )}{L}$$

where L is the distance between the electrodes. It is evident that the Cartesian configuration produces a constant electric field in the treated medium between the electrodes. Identifying the electric field parameters that separate between reversible and irreversible electroporation requires numerous constant electric field experiments, in which the electric field strength is continuously changed in separate experiments until the interface is detected approximately, through interpolation between experiments. (Rubinsky et al. 2008)

Several approaches were introduced for multiparameter optimization of in vitro and in vivo electroporation. Heiser (1999) published an extensive review on electroporation parameters for various cell lines and general guidelines for electroporation protocol optimizations in vitro (Heiser 1999). A review and guidelines for optimization of in vivo electroporation applications was reported on by (Gehl 2003). Furthermore, several statistical methodologies were proposed to reduce the number of experiments required for protocol optimizations. Multifactorial experimental design for optimizing transformation protocols was introduced by (Marciset and Mollet 1994). Keng-Shiang et al. (2007) used the Taguchi Method for the optimization of gene electrotransfer (Keng-Shiang et al. 2007). Recently, a central composite design was used to optimize electroporation protocols (Madeira et al. 2010).

In this study we developed a different approach to multiparameter optimization, based on the use in a single experiment of a well-defined variable electric field topology in the curvilinear coordinate system. The concept will be illustrated with a simple to implement cylindrical coordinate system. The electric field calculated from the solution to the one dimensional Laplace equation in cylindrical coordinates, in a medium between two cylinders of radiuses R₁ and R₂ on which electric potentials of V₁ and V₂ are imposed, respectively, is given by,

$$\frac{( {V}_{2} - {V}_{1} )}{r ln [ \frac{{R}_{2}}{{R}_{1}} ]}$$

where, r is the variable radius inside the domain of interest. Obviously, the electric field varies continuously as an inverse function of the radius. (In one-dimensional spherical coordinates the electric field varies as one over the radius squared). Therefore, in a single experiment in one-dimensional cylindrical or spherical electrode systems, the cells between the electrodes will experience a continuously variable electric field, that is, nevertheless, well defined as a function of the radius. The response of the cells to any electroporation protocol can be evaluated as a function of their relative location (defined by radius) and thereby correlated to the electric field. Therefore, when an experiment is performed with cylindrical (or spherical) electrodes, the results of a single experiment produce continuous information on the effect of a wide range of electric fields, which are quantified by the radius at which they are produced. In contrast, to produce similar information, the conventional Cartesian electrode system requires a very large number of experiments and the interpolation of results between the studied discrete data points.

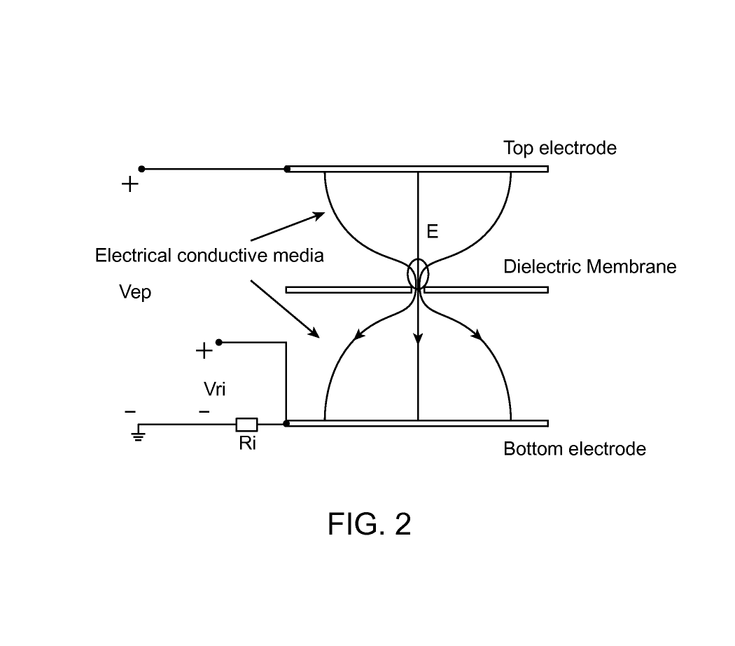

FIG. 2, illustrates results obtained from a study performed with a one-dimensional cylindrical system, using Escherichia coli BL21 (D13) PSJS1244, an ampicillin stable strain. The FIG. 2d shows the electric field at the reversible/irreversible interface as a function of the number of pulses. The microorganisms were spread on a Petri dish and a constant pulsed electric potential was imposed on two concentric metal cylinders, in contact with the surface on which the microorganism was plated. In the one dimensional cylindrical electrode system used, the outer diameter of the inner cylinder was 1.18 mm and the inner diameter of the outer cylinder was 22.15 mm. The electric pulse was delivered by a BTX (BTX ECM 830, Harvard Apparatus, MA). Four sites were treated in each Petri dish, after which the samples were incubated for 18 hours at 37° C. and examined. FIGS. 2a,d reports on results with a pH buffered plate, at which pH did not change after the application of electric field, and in which 2200 V pulses were applied between the concentric electrodes in 40 μs pulse duration at 1 Hz frequency. Five repeats were performed for each condition. It should be emphasized that each data point on the curves was obtained from a single experiment (with five repeats). In contrast, obtaining such a single data point with Cartesian electrodes would require numerous single electric field experiments and interpolation.

FIGS. 2b and 2c show how the plot in FIG. 2d was obtained. FIG. 2a shows the appearance of a treated cylinder after incubation. It is evident that the cells in the central area did not survive the electric fields to which they were exposed and did not form colonies. To determine the radius of cell death we measured the innermost radius of the colonies that survived electroporation, as described in Materials and Methods. FIG. 2b shows the model of the analyzed system. Two cylindrical electrodes, with radiuses of R₁ and R₂, and a measured radius (r) of a zone where irreversible electroporation takes place, are shown on FIG. 2b. Then, the mathematical expression for the electric field as a function of radius in cylindrical coordinates was used to produce FIG. 2c. FIG. 2c was used to correlate the radius of cell death in FIG. 2a with the electric field at that radius. The electric field at the radius of cell death is than plotted as a function of number of pulses in FIG. 2d. This plots the electric parameters at which irreversible electroporation begins.

This work shows that the use of cylindrical one-dimensional electrodes will substantially reduce the number of experiments needed to design optimal electroporation protocols, over those obtained with the use of traditional Cartesian electrodes. The results show that the use of the concept for obtaining the reversible irreversible interface. Obviously a similar experiment with fluorescence dies or genes can be used to determine the parameters at the interface between reversible and no effect electric fields. Furthermore, this method provides a means to examine in a single experiment, various colonies that have undergone electroporation with a wide range of well-defined electroporation parameters. The relative location of each colony of interest identifies the electroporation conditions it has experienced. It should be noted that the idea of a well-defined topological space of variable electric fields could be extended to the design of systems of more complex surfaces than the cylinder or sphere, which may produce in a single experiment complex ranges of parameters of interest.

### Example 1

### Methods and Materials

### Experimental Device

The cylindrical one-dimensional electroporation electrodes were manufactured using a Perspex “square” (3 cm by 3 cm) basis. A half cm notch carved in the side of the square was attached to the top of a brass ring using a heated glue gun. The brass ring had an inside diameter of 22.15 mm, an outside diameter of 25.40 mm, and a height of 4 mm. The tip of an 18 gauge steel needle (Precision Glide needle, Becton Dickinson & Co, NJ) was cut 1 cm from the top, to form the inner, 0.6 mm radius cylinder. The needle was then inserted through the center of the plastic square in the middle of the brass ring forming two concentric cylinders.

### Electroporation Procedure

The study was performed using E. coli BL21 (D13) PSJS1244 an ampicillin stable strain. A single E. coli colony was used to inoculate 50 mL of sterile LB Broth (Ditco, NY) containing 100 μg/mL of ampicillin (American system, CA). The sample was placed in a Thermo Scientific MaxQ 4450 shaker-incubator. The temperature was maintained at 37° C. The shaker speed was 200 rpm to allow aeration for optimal growth. The sample was kept in the shaker-incubator for 14 hours to reach stationary phase. The final concentration of approximately 10⁶ CFU/mL was determined by viable count method. After 14 hours in the shaker-incubator a 100 μL sample was removed and diluted in 10 ml of sterile water (100× dilution) 100 μL of the diluted sample was plated on to each pre-prepared agar plate and spread using glass beads (Novagen, CA). The glass beads were removed and the electroporation device was inserted into the agar in one quadrant of the Petri dish. The device was pushed into the agar plate until the ring and needle touched the Petri dish bottom in order to ensure they were at the same depth. Alligator clips were attached to the brass ring and the 18G needle. The alligator clips were never in direct contact with the agar to ensure no direct discharge into the gel. This allowed the field to be equally distributed around the needle. The clips were hooked up to the BTX (BTX-model 610, BTX ECM 830 square-wave e; electroporator, Harvard Apparatus, MA). The electroporation parameters used were 2200 V, 40 μs pulse duration, 1 Hz frequency. The numbers of pulses were changed between experiments. Statistical analysis was done with the final parameters recorded from the BTX device.

Following the electroporation the needle and the ring were removed from the agar gel. (A similar experiment was than performed in another quadrant. A total of four experiments were performed per dish). A total of five experiments per parameter were performed. After the experiment the Petri dish was incubated at 37° C. for 18 hours. Following the incubation period the dishes were removed and IRE curve radius was measured.

### Plate Preparation

1 g/L NaCl (Spectrum Chemical, Mfg Corp, CA), 10 g/L Bactotryptone, 5 g/L Yeast Extract, 15 g/L Bacto Agar (Becton, Dickison and Company, NJ), 0.5 g/L Glucose was dissolved in distilled water and heated at 121° C. in an autoclave for 15 minutes. After cooling down and reaching 50° C., 23.83 g/L Hepes (Sigma-Aldrich, CA) and ampicillin (American Bioanalytical) at 10 mg/mL was added to 100 μg/mL final concentration. The buffered agar was then poured into a 100 mm Petri dish and the drying time between the pouring and the closing of the plates was 6.5 minutes. In fact, the evaporation of water during storage must be taken into account because it changes the NaCl concentration and of course the conductivity of the medium.

### Radius Measurement and Statistical Analysis

Electroporated plates were removed from the incubator after 18 hours. Digital images of the plates and scale reference were taken and then used to determine the death zone diameter. The error on the electric field estimate includes the diameter measurement errors (precision of 0.05 mm) and the BTX device output error (20 V). The reported radius is an average of five repeats with a Standard Deviation calculated from the five measurements.

### REFERENCES

Dev S, Rabussay D, Widera D, Hofmann G. 2000. Medical applications of electroporation IEEE Transactions on Plasma Science 28(1):206-223.
Gehl J. 2003. Electroporation: theory and methods, perspectives for drug delivery, gene therapy and research. Acta Physiologica Scandinavica 177(4):437-447.
Heiser W C. 1999. Optimizing Electroporation Conditions for the Transformation of Mammalian Cells. In: Tymms M J, editor. Transcription Factor Protocols. Totowa, N.J. Springer. p 117-134.
Keng-Shiang H, Sheng-Chung Y, Hung-Yi C, Yu-Cheng L. Optimization of Gene Transfection Condition using Taguchi Method for an Electroporation Microchip; 2007 10-14 Jun. 2007. p 847-850.
Madeira C, Ribeiro S, Turk M, Cabral J. 2010. Optimization of gene delivery to HEK293T cells by microporation using a central composite design methodology. Biotechnology Letters 32(10):1393-1399.
Marciset O, Mollet B. 1994. Multifactorial experimental design for optimizing transformation: Electroporation of Streptococcus thermophilus. Biotechnology and Bioengineering 43(6):490-496.
Neumann E, Schaefer-Ridder M, Wang Y, Hofschneider P H. 1982. Gene transfer into mouse lyoma cells by electroporation in high electric fields. EMBO Journal 1(7):841-845.
Pakhomov A G, Miklavcic D, Markov M M. 2010. Advanced Electroporation Techniques in Biology and Medicine. Boca Raton: CRC Press 521 p.
Rubinsky, B. Ed. “Irreversible Electroporation” Springer. Series in Biomedical Engineering 2010, XIV, 314 p., Hardcover Chemai ISBN: 978-3-642-05419-8
Rubinsky J, Onik G, Mikus P, Rubinsky B. 2008. Optimal Parameters for the Destruction of Prostate Cancer Using Irreversible Electroporation. Journal of Urology 180(6):2668-2674.
Sale A J H, Hamilton W A. 1967. Effect of high electric field on micro-organisms. I. Killing of bacteria and yeast. Biochimica et Biophysica Acta 148:781-788.
Hamilton W A, Sale A J H. 1967. Effect of high electric field on micro-organisms. II. Mechanism of action of the lethal effect. Biochimica et Biophysica Acta 148:788-800.
Stopper H, Zimmermann U, Wecker E. 1985. High yields of DNA-transfer into mouse L-cells byelectropermeabilization. Zeitschrift fur Naturforschung 40(11-12):929-932

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

That which is claimed is:

1. A method of directing an electric field at a targeted area of cells, comprising:
determining an area of cells to be targeted;
positioning a shaped metal surface into contact with the area of cells to be targeted;
contacting a first and a second electrode with the shaped metal surface;
generating an electrical charge differential between the first and second electrodes;
wherein the electrodes are shaped and positioned on the metal surface in a manner such that an electrical field generated by the charge differential and emitted from the metal surface matches the shape of the area of cells to be targeted.

2. The method of claim 1, wherein the metal surface is comprised of a metal selected from the group consisting of copper, silver, gold and combinations thereof.

3. The method of claim 1, wherein the electrical charge differential is provided within defined ranges of voltage, wattage and duration.
